(19) United States
(12) Patent Application Publication
(10) Pub. No.: US 2016/0007834 A1
(43) Pub. Date: Jan. 14, 2016

(54) ENDOSCOPE

(71) Applicant: OLYMPUS WINTER & IBE GMBH, Hamburg (DE)

(72) Inventor: Uwe SCHOELER, Hoisdorf (DE)

(73) Assignee: OLYMPUS WINTER & IBE GMBH, Hamburg (DE)

(21) Appl. No.: 14/769,617

(22) PCT Filed: Jan. 6, 2014
(86) PCT No.: PCT/EP2014/000007

§ 371 Date: Aug. 21, 2015

(30) Foreign Application Priority Data
Feb. 28, 2013 (DE) 10 2013 003 315.2

(51) Int. Cl. A61B 1/00; B23K 26/21; B23K 26/32; A61B 1/005

(52) U.S. Cl. CPC A61B 1/00128; A61B 1/005; A61B 1/0011; A61B 1/00078; B23K 26/21; B23K 26/32

(57) ABSTRACT

An endoscope including: a flexible portion having an elastic hose; at least one rigid portion having at least at one end adjoined to the flexible portion at an end area, the at least one end being enclosed by the elastic hose; and a clamping device disposed over the elastic hose at the end area to sealingly clamp the flexible portion to the at least one rigid portion by applying a radial force to the end area, the clamping device including a loop member which is made of metal and which winds around the hose with at least two windings, and in which at least the two windings are welded to one another.

### CROSS-REFERENCE TO RELATED APPLICATION

The present application is based upon and claims the benefit of priority from PCT/EP2014/000007 filed on Jan. 6, 2014, which claims benefit to DE 10 2013 003 315.2 filed on Feb. 28, 2013, the entire contents of each of which are incorporated herein by reference.

### BACKGROUND

1. Field

The present invention generally relates to endoscopes, and more particularly to an endoscope of the type referred to in claim 1 and also to a method for its production.

2. Prior Art

In addition to rigid endoscopes, often enclosed by metal tubing, such as are used in urology and laparoscopy; generic endoscopes are known which have rigid and flexible portions that adjoin one another. Even endoscopes that are likely designated as completely flexible, such as those for intestinal examinations, are formed to be at least rigid on their proximal handling part, and have at that point a transition from a flexible to a rigid portion. In this case, the outer seal of the endoscope is of decisive importance, not only for endoscopes used in industrial applications, but in particular for medical endoscopes.

The outer seal is then particularly important when the endoscope is being prepared. Preparation is understood to include cleaning and sterilization, thus activities that use liquids and e.g. also steam, from which the complicated inner workings of the endoscope, with its optical and electronic components, should be protected.

Flexible portions of endoscopes have internally at most one stiffening framework, which consists of parts flexibly connected to one another or of helical spring type elements. An elastic hose, which guarantees the fluid-tight seal, always surrounds these flexible portions on the outside.

The problem thereby is the sealing of the ends of the hose, which usually takes place generically by pushing the hose on and radially clamping it to the rigid portion. This displaces the entire sealing problem to the correct action of the clamping device.

Clamping devices in the form of conventional hose clamps, as are common in many branches of technology, cannot be used here, as outwardly protruding and potentially sharp-edged parts are not allowed for use in a patient's body.

A known solution consists in binding off the hose pushed onto the rigid portion using a thin plastic thread, similar to fishing line; the thread is tightened and the ends are knotted. The clamping device is subsequently completed by a cover with the aid of an adhesive material, a lacquer, or by using an adhesive tape. This construction provides good results for the user; however, there are also disadvantages during production. A mechanical production of the clamping device is not possible. It can only be completed by people with very dexterous fingers. High production costs result from this.

### SUMMARY

An object consists in making an endoscope of the type listed in the beginning more economically with high safety.

This problem is solved using the features of claim 1, and by a method according to claim 6.

Accordingly, the endoscope uses a metal loop member, in which windings are welded. A clamping device of this type can be attached mechanically with a low error rate. By this means, costs are reduced and reliability is increased, thus in particular, patient safety in the case of medical use.

Advantageously according to claim 2, an eyelet is provided on the end of the loop member. By means of this, a loop may be very easily formed, which facilitates the start of the looping process.

Advantageously according to claim 3, the loop member is formed as a flat strip. This enables a high strength at low thickness and easy and secure welding across the width of the flat strip. In addition, the risk of injury is reduced due to the flat configuration.

Advantageously according to claim 4, the flat strip is broadened in the area of the eyelet. This provides a high edge stability for the eyelet and the possibility of welding the flat strip, which returns here after wrapping the hose over the eyelet, wherein the broadened area protects the hose from the effects of the welding process.

Advantageously according to claim 5, the loop member is formed as wire. By this means as well, advantageous embodiments of the clamping device may be created which have no sharp-edged points and are able to be safely welded, without damaging the hose.

The production of the endoscope takes place advantageously using the feature of one of claims 6 through 8.

### BRIEF DESCRIPTION OF THE DRAWINGS

In the drawings, the invention is presented schematically and by way of example.

FIG. 1 illustrates a side view of an endoscope with two clamping devices,

FIG. 2 illustrates an enlarged section through the endoscope from FIG. 1 in the area of a clamping device with a loop member,

FIG. 3 illustrates a loop member from another embodiment during the forming of a loop,

FIG. 4 illustrates the loop member from FIG. 3 after completed placement and welding, and

FIG. 5 illustrates a greatly enlarged side view of the endoscope from FIG. 1 in the area of a clamping device from another embodiment.

### DETAILED DESCRIPTION

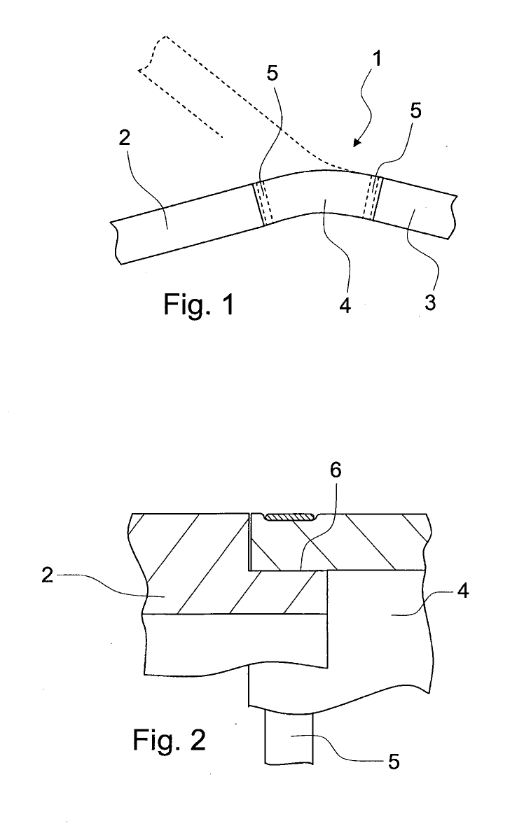

FIG. 1 shows in side view a longitudinal section of a shaft of an endoscope 1. The proximal connection and handling portion and also the distal end portion are omitted. Endoscope 1 as shown may be an industrial or a medical endoscope which may accommodate in its interior (not shown) image guides, electrical lines, gas and/or fluid channels, and other equipment.

In the area shown, endoscope 1 includes three portions, namely a rigid portion 2 and a further rigid portion 3. These may both be formed e.g. as steel tubing on their depicted outer sides.

A flexible portion 4 is arranged between the two rigid portions 2 and 3. Flexible portion 4 is formed on its outer side by an elastic hose which enables bending of flexible portion 4, as is indicated in FIG. 1 by the dashed lines represented a second bent position.

Clamping devices 5 are arranged on the two end areas of flexible portion 4 (represented as dashed lines in FIG. 1). FIG. 2 shows an enlarged section through a portion of the structure shown in FIG. 1 in the area of the clamping device abutting rigid portion 2.

FIG. 2 shows rigid portion 2, which is formed as metal tubing, at which the end area 6 abutting flexible portion 4 is reduced in diameter. With regard to flexible portion 4, only the elastic hose is shown here. The entire inner workings of flexible portion 4 are omitted. The hose is pushed over rigid portion 2, namely over its reduced end area 6, by which means a smooth transition between hose 4 and rigid portion 2 results on the outer side.

In the end area of hose 4, which sits on reduced end area 6 of rigid portion 2, the clamping device 5 is placed externally around hose 4, which clamping device is shown schematically in FIG. 2 as encircling, strip-shaped loop member.

Clamping device 5, in the form of loop member (referred to herein also with reference numeral 5) is tightened with force around elastic hose 4 and presses itself, as shown in FIG. 2, into the surface of the hose, in order to thus guarantee a long-lasting clamping tension between the inner side of hose 4 and the outer side of rigid portion 2 in end area 6. From this, a good sealing effect results due to the elastic material design of hose 4.

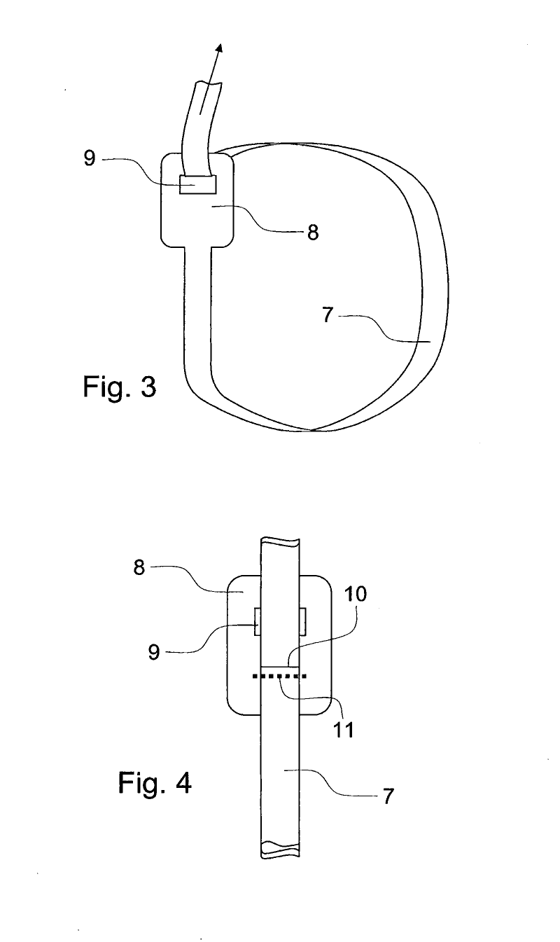

FIGS. 3 and 4 show a first specific configuration of the clamping device. The loop member is thereby formed of metal as flat strip 7, which has an eyelet 9 on one end in a broadened area 8.

The structure from FIG. 3 may be placed on one of the two placement points for the clamping device shown in FIG. 1. Flat strip 7 is thereby wound around hose 4 and inserted through eyelet 9. When pulling on the free end of flat strip 7 in the direction of the arrow as shown, the loop shown in FIG. 3 is drawn around hose 4 until it firmly encircles the same in the end position in this way, as this is shown in FIG. 2.

This results in the placement position of the clamping device according to FIG. 4, wherein in the embodiment shown in FIG. 4, the clamping device is shown with three windings of flat strip 7 lying superposed; the top and bottom of the windings are respectively truncated.

The first winding is shown in FIG. 3. Flat strip 7, which is drawn in the direction of the arrow through eyelet 9, is wound in a second and third winding lying superposed around the hose of flexible portion 4. The third winding ends at line 10 of FIG. 4. The strip is pulled tight, until, as indicated in FIG. 2, it abuts with a high force. The fastening is then carried out by welding different windings of flat strip 7, which consists of suitable, easily weldable metal. FIG. 4 shows a welded seam 11 which is drawn for example transversely over the outermost winding of metal strip 7 up to the adjacent broadened area 8. Welded seam 11 thus connects the first winding with the third. It may also be welded through in such a way that all windings are welded to one another. As shown in FIG. 4, welded bead 11 may easily be drawn laterally across strip 7 onto broadened area 8 without damaging the material of hose 4.

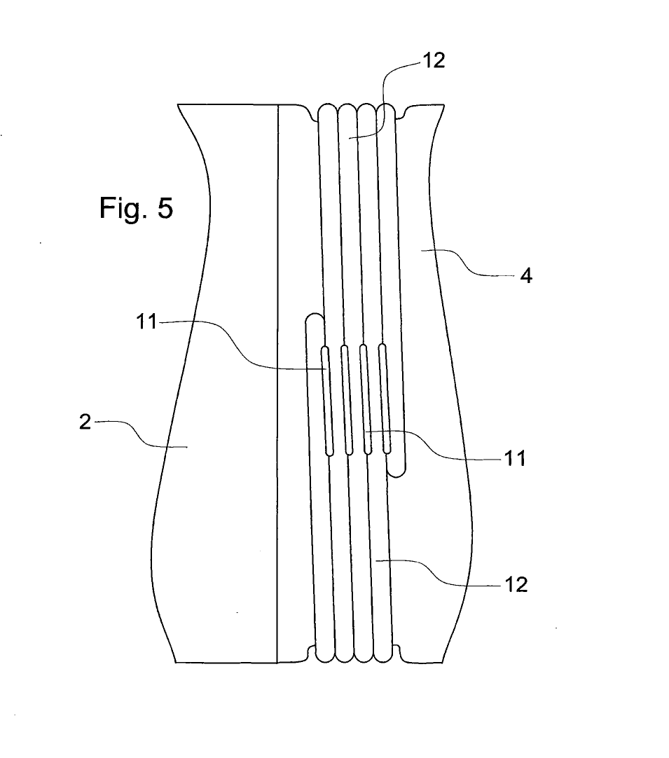

FIG. 5 shows an alternative embodiment of the clamping device, namely at the point of the clamping of elastic hose 4 onto rigid portion 2, which may be configured in profile view exactly as is shown in FIG. 2. The clamping device is, however, configured differently than in FIGS. 3 and 4, where the loop member of the clamping device is configured as flat strip 7. In the embodiment of FIG. 5, the loop member is, in contrast, formed from a metal wire 12.

In contrast to the embodiments of FIGS. 3 and 4, in which the windings of flat strip 7 are wound superposed, multiple windings of metal wire 12 are wrapped next to one another in the embodiment of FIG. 5. In the embodiment shown in FIG. 5, there are 4 windings.

Using a suitable winding machine, the metal wire, as shown in FIG. 5, is wound multiple times around hose 4 and drawn tight with great force, as FIG. 5 also shows by means of the effect on hose 4. Metal wire 12 is then held in the tightly wound position and fixed by welding. This takes place as is shown in FIG. 5.

In this case, one welded seam 11 is always arranged between two windings. In total, there are thus four welded seams. These are arranged on a relatively short circumferential area along the contact line between each two windings. If the windings are wrapped sufficiently closely, then they may be welded here without damaging the hose lying below them.

Deviating from the embodiment of welded seams 11 shown, an arrangement of welded seams in the direction of the axis of the endoscope, thus essentially transverse to the placement of the welded seams shown in FIG. 5, may also be used.

As in the embodiment of FIGS. 3 and 4, the welded seams can be carried out using a welding laser, which is sufficiently narrowly focusable using suitable optics in order to be able to cleanly produce narrow welded seams, as are also shown greatly enlarged in FIG. 5. In the actual embodiment, for example the diameter of the endoscope shown is only approximately 5 mm. Metal wire 12 would then have a thickness of 0.05 through 0.2 mm and welded seams 11 would have a width of approximately 0.05 mm.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. An endoscope comprising:
a flexible portion having an elastic hose;
at least one rigid portion having, at least at one end adjoined to the flexible portion at an end area, the at least one end being enclosed by the elastic hose; and
a clamping device disposed over the elastic hose at the end area to sealingly clamp the flexible portion to the at least one rigid portion by applying a radial force to the end area, the clamping device including a loop member which is made of metal and which winds around the hose with at least two windings, and in which at least the two windings are welded to one another.

2. The endoscope according to claim 1, wherein the loop member is provided with an eyelet on one end.

3. The endoscope according to claim 2, wherein the loop member is formed as a flat strip.

4. The endoscope according to claim 3, wherein the flat strip has a broadened width, relative to other portions of the flat strip, in the area of the eyelet.

5. The endoscope according to one of claim 1, wherein the loop member is formed as a wire.

6. A method for producing the endoscope of claim 1, the method comprising:
pushing the hose with the end area over the at least one rigid portion;
looping the looped member at least once around the end area;
tightening the loop member to apply the radial force to the end area, and
welding the at least two windings of the loop member to one another.

7. The method according to claim 6, wherein the welding comprises laser welding the at least two windings to one another.

8. The method according to one of claim 6, wherein the loop member is provided with an eyelet on one end and the tightening comprises inserting the loop member through the eyelet.

9. (canceled)
